(19) United States
(12) Patent Application Publication
(10) Pub. No.: US 2022/0111083 A1
(43) Pub. Date: Apr. 14, 2022

(54) PORTABLE SYSTEMS AND METHODS FOR DISINFECTING OBJECTS USING LIGHT SOURCES

(71) Applicant: Sterling E. Johnson, Los Angeles, CA (US)

(72) Inventor: Sterling E. Johnson, Los Angeles, CA (US)

(21) Appl. No.: 17/066,464

(22) Filed: Oct. 8, 2020

(51) Int. Cl. A61L 2/10; B65D 21/08

(52) U.S. Cl. CPC A61L 2/10; A61L 2202/16; A61L 2202/122; B65D 21/086

(57) ABSTRACT

The present invention discloses novel ultraviolet collapsible frameworks (UCF's) that, when housed in correspondingly expandable and collapsible containers, enable the design of UV disinfecting devices that are easy to use, safe, relatively lightweight, portable and easy to store, and that can effectively disinfect a wide range of objects. In some embodiments, replaceable UVC emitter pods are powered by pod stations disposed on the framework structure.

### RELATED APPLICATIONS

None.

### TECHNICAL FIELD

The present invention relates generally to the field of object disinfection and in particular to systems and methods for disinfecting objects using light sources.

### BACKGROUND

Rigorous laboratory testing and biological studies have proven the effectiveness of ultraviolet (“UV”) radiation, and in particular UV-C radiation (hereinafter “UVC”, often defined as, but not strictly limited to, light in the 200 nm to 280 nm range), in destroying dangerous pathogens that are found in the environment, including: Norovirus, E. Coli, Salmonella, Staphylococcus, Bacillus, Tuberculosis, Candida, among many others. Indeed, UVC is used extensively to disinfect air, water and surfaces in order to help mitigate the risk of people acquiring these and other infections. In the current environment, UVC has been increasingly used to disinfect hospitals, airplanes, and other public spaces to combat the spread of Covid-19 and other harmful viruses and bacteria. See, for example, https://www.reuters.com/artide/us-health-coronavirus-germfalcon/coronavirus-pandemic-inspires-demand-for-uv-airplane-cleaner-idUSKCN2263AC. As far back as 2001, the FDA approved the use of UVC lamps use in commercial food production to cold pasteurize liquids and to prolong the shelf life of food. See, for example, http://artide.sapub.org/10.5923.j.food.20120201.03.html. Thus, the prospect for the beneficial use of UVC has long been recognized.

However, the use of UVC presents a number of challenges. First and foremost, exposure to UVC radiation can be dangerous to humans. Without protections and precautions such as safety gloves, special glasses, distance from the source, etc., UVC can damage DNA—precisely why it is an effective disinfectant. See, for example, https://www.ncbi.nlm.nih.gov/pmc/articles/PMC3010660/. Despite the danger, portable UVC “wands” are available for consumers to purchase and use at their own risk. Many of these devices come accompanied with a flimsy warning instructing the user to use special goggles and gloves to limit exposure.

Moreover, the high UVC dosage needed to effectively disinfectant many objects (a function of time, distance, and output/power of the UVC light source) greatly increases the safety hazard and potential for user error. These inherent safety hazards create a need for better solutions for containing harmful UVC radiation designed for disinfecting objects by creating an effective, user friendly solution that substantially reduces the safety risks and concerns and the inevitable human error associated with exposure to uncontained UVC radiation.

A number of fully enclosed UV disinfection device designs exist for the purpose of safely disinfecting objects of limited size. These devices are similar to the microwave machine in shape and size. They are typically designed to disinfect baby bottles, manicure tools and food. Unfortunately, these rigid boxes are relatively large and heavy, and are not amenable to easy movement or storage. For that reason, they have limited utility. On the other end of the spectrum, small, rigid, and portable UV boxes do exist. However, they are typically designed to disinfect small objects like mobile devices and keys, and thus the utility of these devices is limited by their diminutive size. Examples of prior art devices that suffer from one or more of the aforementioned and other drawbacks include those disclosed in the following US patents. U.S. Pat. No. 8,536,541 discloses a rigid box that is not collapsible/storable/portable. Published US application no. US20130063922 discloses a small UV clamshell design that does not accommodate items much larger than a mobile phone. U.S. Pat. No. 9,463,890 discloses a number of UVC light sources that rely on reflection, resulting in diminished efficacy. U.S. Pat. No. 9,066,987 contemplates a delicate multilayered UVC LED substrate that is not commercially viable. And, U.S. Pat. No. 8,662,705 discloses a flexible ultraviolet LED sanitizing apparatus. But this disclosure does not allow for the replacement of individual UVC light sources.

There thus exists a need for an effective UV disinfecting system and structure that overcomes the limitations of the prior art. Such a system would safely disinfect a wide array of objects that may be much larger than mobile devices, while still being lightweight, portable and long-lasting. The present invention meets these needs and more.

### SUMMARY

The present invention meets these needs and more by disclosing ultraviolet collapsible frameworks (UCF's) having size, shape, and design profiles that enable the application of UV disinfection to a wide range of objects and use cases. The inventive UCF concept enables the design and development of inventive disinfection devices having containers of various shapes that are easy to use, safe, relatively lightweight, portable and easy to store. The present invention's preferred use of strategically oriented disinfecting UVC light sources as part of the inventive framework can maximize surface area exposure of items to be disinfected for maximum disinfection coverage while minimizing exposure times needed for effective disinfection. The sizes of the inventive UCF framework can range widely to accommodate and sanitize a variety of commonly used items—from electronic devices, masks, keys, food delivery containers, and groceries, to much larger packages and items such as pillows, as well as to disinfect several smaller items at once. Further, in some embodiments, the UCF's light sources may be easily removed and replaced by the user if a light segment is damaged or burns out.

The present invention also discloses portable UVC containers that incorporate the inventive frameworks disclosed herein. Theses containers may be made of pliable or soft material that house the frameworks, such that when not in use the entire containers can be collapsed into a small area for easy storage and transport, in some cases using the lid of the device as the storage container.

It is to be understood that the present invention is not limited in its application to the details of construction and the arrangement of components described hereinafter and illustrated in the drawings and photographs. Those skilled in the art will recognize that various modifications can be made without departing from the scope of the invention.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1a is a diagrammatic perspective view of a portable UVC disinfecting apparatus according to the present invention, shown in a non-limiting collapsible, helical coil framework embodiment;

FIG. 1b is a diagrammatic perspective view of the apparatus of FIG. 1a shown with the container in a collapsed state with its cover in position to receive the container;

FIG. 1c is a diagrammatic perspective view of the apparatus of FIG. 1a shown in a fully collapsed and closed state;

FIG. 2 is perspective view of one specific embodiment of a UVC collapsible framework (UCF) as may be used in the apparatus shown in FIG. 1;

FIG. 3a is a diagrammatic top view of a pod station as may be used in the UCF of FIG. 2;

FIG. 3b is a diagrammatic top view of a UVC pod that may pair with the pod station shown in FIG. 3a;

FIG. 4a is a diagrammatic side view of a UVC pod and pod station as shown in FIGS. 3a and 3b in position to be assembled together;

FIG. 4b is a diagrammatic side view of a UVC pod pair as shown in FIG. 4a as assembled;

FIG. 5a is a diagrammatic perspective view of an alternative embodiment apparatus of the present invention, wherein the UVC framework is a square, collapsible scissor framework;

FIG. 5b is a diagrammatic perspective view of the apparatus of FIG. 5a shown with the framework and container in a collapsed state;

FIG. 5c is a diagrammatic perspective view of the apparatus of FIG. 5a and shown in a fully collapsed and closed state;

FIG. 6a is a diagrammatic perspective view of an alternative square UV collapsible container apparatus according to the present invention; and

FIG. 6b is a diagrammatic perspective view of the apparatus of FIG. 3a shown in a fully collapsed and closed state.

### DETAILED DESCRIPTION OF THE PREFERRED EMBODIMENTS

The present invention may be implemented in many different embodiments. In one, a collapsible, UV disinfecting framework, may include an expandable and collapsible framework structure defining, when in an expanded state, an inner space into which an object for disinfection may be placed; and a UV-emitting light source attached to the framework structure, the source positioned such that, when electrically powered, directs ultraviolet energy toward the inner space. In some embodiments, the framework structure may be a helically shaped compression spring, and in others it may comprise at least one collapsible, hinged lattice structure for assembly on an inner wall of a collapsible container, or even four (4) collapsible, hinged lattice structures, each for assembly on an inner wall of a four walled, substantially square, collapsible container.

The UV-emitting light source may comprise at least two LED UVC radiating emitters which may be designed as a string of electrically connected UVC emitters that is disposed along at least a portion of the framework structure. The UV-emitting light source may also be designed as one or more UV pod/pod station pairs. In this embodiment, the at least one pod station may be connected to the UV collapsible framework structure and the UV pod may be removably connected to the pod station, such that when powered, the pod station supplies electrical power to the connected pod. Thus, in a more particular embodiment, at least two pod stations may be connected to the framework structure and a respective UV pod may be removably connected to each pod station, such that when powered, each pod station supplies electrical power to its connected UV pod.

In yet another embodiment, a sealable and portable UV disinfecting device is disclosed having a framework with a vertically expandable and collapsible framework support that when in an expanded state defines an inner space into which an object for disinfection may be placed, and an electrically powered UV emitter attached to the support that when powered directs energy toward the inner space. It may also have a sealable container that contains the framework and that is collapsible with the framework support.

In a further embodiments of the invention, a portable UV disinfecting device may include a helically shaped UV disinfecting collapsible framework having an expandable and collapsible framework structure defining, when in an expanded state, an inner space into which the object may be placed, and a UV-emitting light source attached to the framework structure, with the source positioned such that, when electrically powered, directs ultraviolet energy toward the inner space. It may also include a cylindrically shaped collapsible container that houses the framework and that is supported by the framework, the container having a closed bottom end and an open top end. Finally, the device may have a lid for sealably closing the open end of the container after the object to be disinfected is placed within the container. The lid may preferably include its own UVC radiating emitter attached to its inner side, and positioned such that, when electrically powered, directs ultraviolet energy toward the inner space, giving more UV coverage to the inner space. Indeed, in a further optional enhancement, the device may further include a platform placed at the bottom of the container onto which the object to be disinfected may be placed. This platform may itself include a UVC radiating emitter positioned such that, when powered, directs is ultraviolet energy upward toward the inner space. It is understood that the collapsible framework and container may take other shapes.

Methods for disinfecting objects with UV radiation are also disclosed. In one embodiment, the method may include first expanding a collapsed, open-ended, UVC disinfecting device that includes a framework having an expandable and collapsible framework structure defining, when in an expanded state, an inner space, and a UV-emitting light source attached to the framework structure, with the source positioned such that, when electrically powered, directs ultraviolet energy toward the inner space, and a collapsible container that houses the framework and that has an open end. The method then includes the steps of placing the object inside the inner space of the device, sealing the device with a lid place over the open end of the container; and electrically powering the UV-emitting light source. The method may include powering the source for a minimum amount of time that may be deemed sufficient to radiate and disinfect an object placed therein, such as at least 30 second, at least 1 minute or at least 2 minutes. In other embodiments, the source may be powered for a preset or predetermined amount of time that can be automatic, or set by a user of the device.

Referring now to the drawings, like reference numerals designate identical or corresponding features throughout the several views.

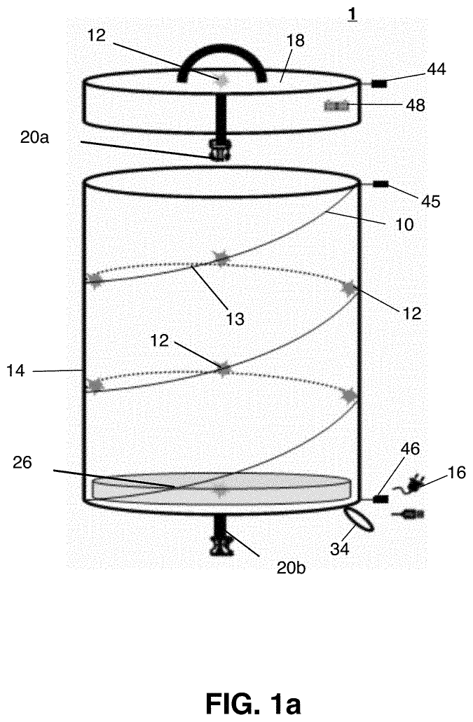

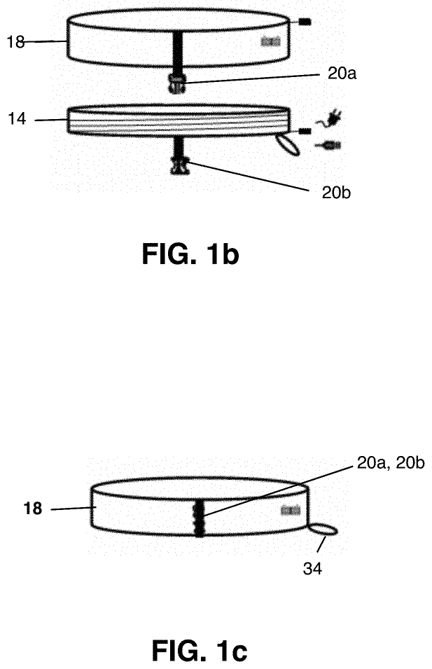

FIGS. 1a, 1b and 1c show a portable, UV disinfecting device 1 accordingly to one preferred embodiment of the present invention. Device 1 includes a compressible helical coil framework structure, or spine, 10 that is biased in an uncompressed state and defines a inner space into which an object for disinfection may be placed, a set of UVC LED sources 12 attached to spine 10 at strategically placed points, container 14 comprising a bottom and a collapsible cylindrical wall that houses and is supported by spine 10, a power source symbolically shown at 46 that, in this embodiment, is removably connectable with a wall plug 16 or USB or other power source to power LED's 12 on spine 10, and a cover or lid 18, shown here lifted off container 14. Also included in this embodiment is platform 26 that rests at the bottom of container 14, and onto which objects for disinfection may be placed. Platform 26 may preferably be a mesh grate made of lightweight aluminum or similar material. The platform's diameter is preferably slightly smaller than the diameter of the container itself, which allows the spine 10 to coil around the platform when the container is collapsed.

As further seen in FIG. 1a, lid 18 and the bottom of container 14 may each include one or more UVC LED sources 12 as well. Thus, as can be appreciated, when operating device 1 on an object placed in it, resting on platform 26 and with the cover closed, LED's 12 may radiate on and disinfect the object from all around, above and below it to achieve maximum disinfection coverage to the object's surface area.

In the embodiment shown, coil spine 10 is a wireframe that preferably acts as a helical spring biased in an “open” position, such that some—although preferably light—force is required to compress it, and thus collapse container 14 for closure. FIG. 1b shows device 1 with container 14 after such downward force has been applied to it, and lid 18 in position to fully cover/enclose the collapsed container 14 and secure it closed with mating closure buckles 20a and 20b. FIG. 1c thus shows the device in its fully closed position after snapping buckles 20a, 20b together. Now, previously large-volume disinfecting device 1 is ready for easy transport, storage and, if desired, hanging via loop 34. When ready to use, the user simply unbuckles closure pair 20a, 20b, and removes lid 18. The container in this embodiment will then “pop up,” creating the cylindrical volume into which to place objects to be disinfected.

Spine 10 with multiple UVC LED sources 12 attached thereto comprises what the inventor of the present invention refers to as ultraviolet collapsible framework (UCF) 13. It should be understood that according to the present invention, UCF's need not take a helical coil shape and need not be biased to pop up into the “open” position. UCF's taking other shapes and designs, some of which are disclosed below, may be used implemented within the scope of the present invention.

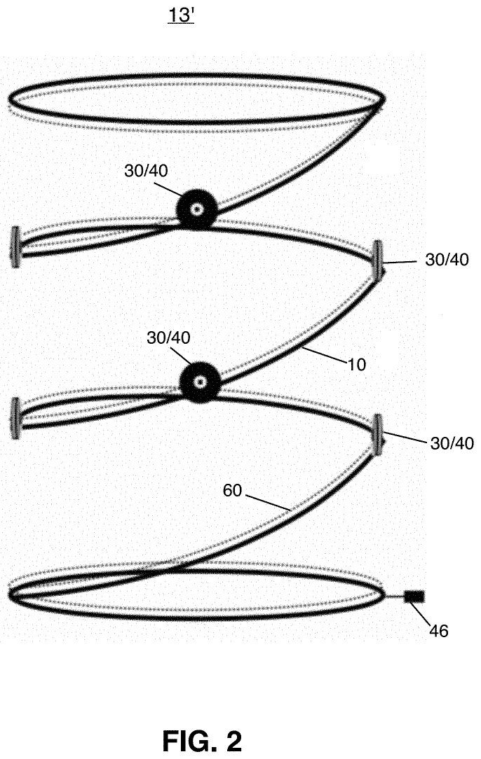

Options for UCF designs will now be discussed. As seen in FIG. 1, framework 13 comprises wireframe spine 10 with UVC LED sources 12 disposed along spine 10 generically shown as starbursts to graphically indicate that when apparatus 1 is powered on, the sources emit disinfecting UVC light. FIG. 2 diagrammatically shows a specific embodiment of UCF 13 in FIG. 1, namely UCF 13′ wherein the UVC sources 12 in FIG. 1 are UVC pod/pod station assemblies 30/40 that are disposed on spine 10. As seen, pod stations 40 are electrically connected together and to power source 46 by wiring, indicated by the dotted line 60 that travels along and is secured to spine 10. As seen, pods 30 are positioned on spine 10 so that their disinfecting light radiates toward the middle of the cavity defined by the spiral spine 10. In this embodiment, a pod 30 may be removably connected to a pod station 40, which supplies power to the pod.

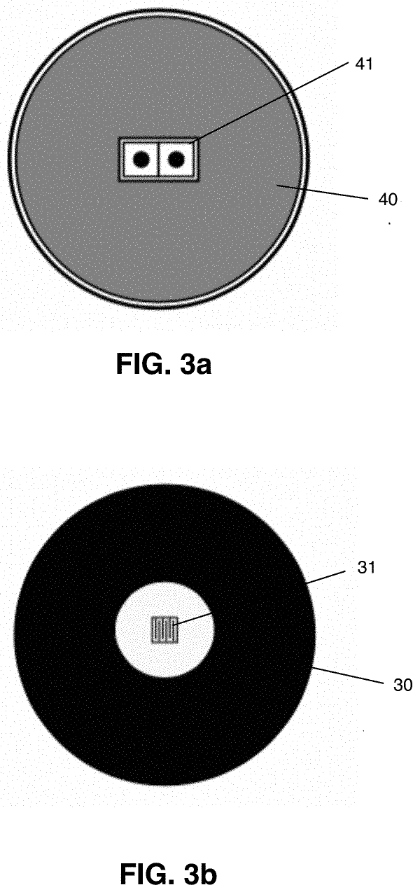

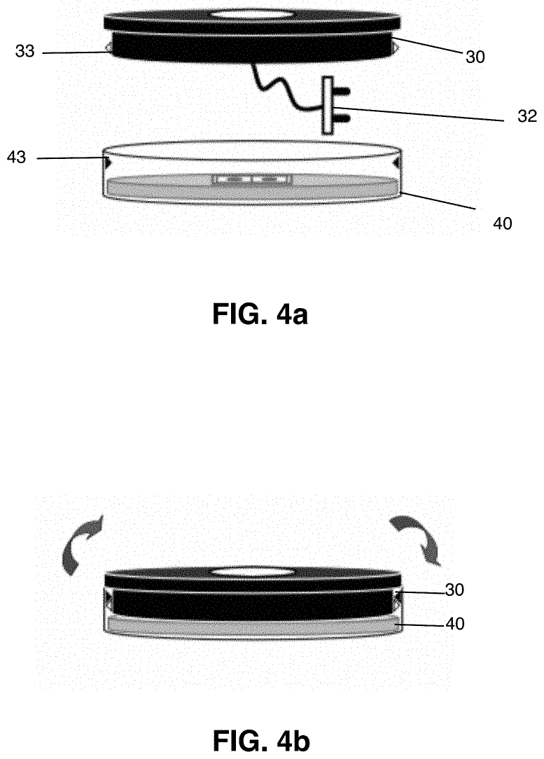

FIGS. 3a, 3b, 4a and 4b diagrammatically show some details of one embodiment of UVC pod/pod station assembly 30/40 shown in FIG. 2. FIG. 3a shows a top view of pod station 40 having a female electrical connector 41 for receiving a plug 32 from pod 30. FIG. 3b shows a top view of LED pod 30 designed in this embodiment with a single UVC LED chip 31. FIG. 4a shows a side view of a pod 30 over and in position to be physically and electrically connected to pod station 40. White triangle 33 on pod 30 and black triangle 43 on pod station 40 represent mating threading, such that after inserting plug 32 of pod 30 into receiving outlet 41 of pod station 40, pod 30 may be secured into pod station, whether by screwing or snapping or other removably attachable means. Thus, in this non-limiting embodiment, FIG. 4b is a side view showing pod 30 and pod station 40 as assembled by screwing pod 30 into station 40 in the directions shown by the arrows.

Thus, turning back to the conceptual embodiment shown FIG. 2, UCF 13′ includes six (6) UVC LED pod/pod station assemblies 30/40 along the spiraling spine 10. If and when any LED pod 30 stops working (whether from burning out or otherwise malfunctioning), it may simply be removed from its station 40 and replaced with a new pod 30.

It should be understood that the number of LED's or LED pods 12 sufficient to ensure adequate, but not too much, UV radiation coverage will vary with a number of factors, including the size and volume of device (e.g., container 14 of device 1 in FIG. 1) and the specifications of the LED and pod design used, including but not limited to its size, power output and angular range of the radiation coverage, to name a few. Pod stations 40 are preferably electrically connected to each other in a “daisy-chain” string similar to Christmas lights, such that supplying power to one end of the pod string i.e., via plug 16, will supply power to all pod stations 40 on the spine.

In other embodiments, the UV collapsible framework (UCF) of the present invention may comprise a single electrically connected string of unreplaceable LED's or LED pods 12, such that when one light stops working the string must be replaced. However, preferably, similar to LED lights found in conventional Christmas light strings, LED's or LED pods may be replaceable when they burn out or malfunction. Moreover, should one pod on a string stop working, it preferably will not create an open circuit preventing the rest of the pods from working. It should also be understood that any convention electrical safety features may be incorporated into the design of any embodiment of the present invention.

Cylindrical container 14 may be made of any suitable collapsible material, such as canvas or flexible polymer material. The interior of the container has a preferably reflective, UV-resistant surface. One such surface may be a coated foil that is able to withstand degradation from repeated exposure to the UV radiation. At the same time, this inner surface may also contribute to the disinfection properties of device 1—namely, its disinfection efficiency. In particular, the reflective coating on the inner wall of container 14 may reflect UVC energy incident on it back toward the middle of the container. This feature may reduce the power output needed from LED's or LED pods or even the number needed to effectively disinfect an object of given size in a given sized container 14.

As noted above, lid 18 may have one or more LED's or UVC LED pods incorporated therein. They may be powered by an electrical plug 44 that connects to top of the container, along with an electronic on/off timer 48. Plug 44 may mate with plug 45 exiting container 14 near its top. The lid preferably has a 3-4 inch “skirt” that hangs down along the outer walls of the container to prevent UV light from escaping. The skirt of the lid is approximately the same height as the container (when collapsed) and connects to the bottom container with a plastic side-squeeze clip 20b creating a one cohesive, interlocked wheel-shaped unit that can be stored or hung on the wall on the small loop 34 at the base of the container.

It should be understood that UV collapsible framework of the present invention can take any shape that (a) creates an inner volume into which an object for disinfection can be placed, and (b) can be inserted/installed inside a collapsible container (taking any shape) that can be fully closed and prevent UV radiation from escaping during operation. Accordingly, the portable UVC disinfecting device of the present invention may advantageously be made in many different sizes and shapes.

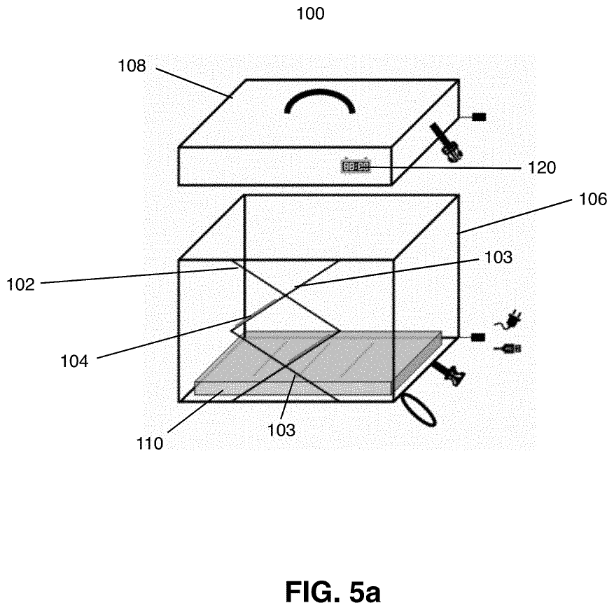

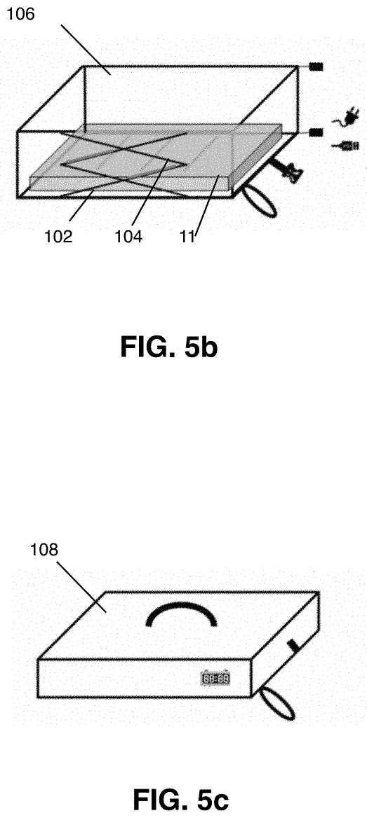

Thus, as seen in FIGS. 5a-5c, the present invention may be implemented in a UV box device 100 having a scissor-shaped UV Collapsible Framework (UCF) 102. In this embodiment, UCF 102 comprises a lattice framework structure on one or more walls of square, or rectangular, collapsible container 106. As shown for just one wall of the container 106, each lattice structure comprises straight pieces 103 that are collapsibly-hinged at various points on the lattice. UV LED strips 104 may be disposed on one or more pieces of the structure. Alternatively, UV lamps may be implemented. In one preferred embodiment of collapsible square box device 100, a UV lattice structure is disposed on each of the 4 inner walls of container 106. As in the prior embodiment, UCF 102 serves the dual role of providing the pop-up/collapse mechanism for the device as well as providing the UV radiation for objects placed on platform 110. Device 100 also includes lid 108 that covers the opening at the top of box 106, as well as the power and buckling features described in connection with the prior embodiment. FIG. 5b shows device 100 in a collapsed state and before lid 108 is placed on and secured to it. As seen, when collapsed, UCF 102 compresses down in an accordion-like fashion, at which point, as seen in FIG. 5c lid 108 may be placed over container 106, secure closed and store and transported as needed.

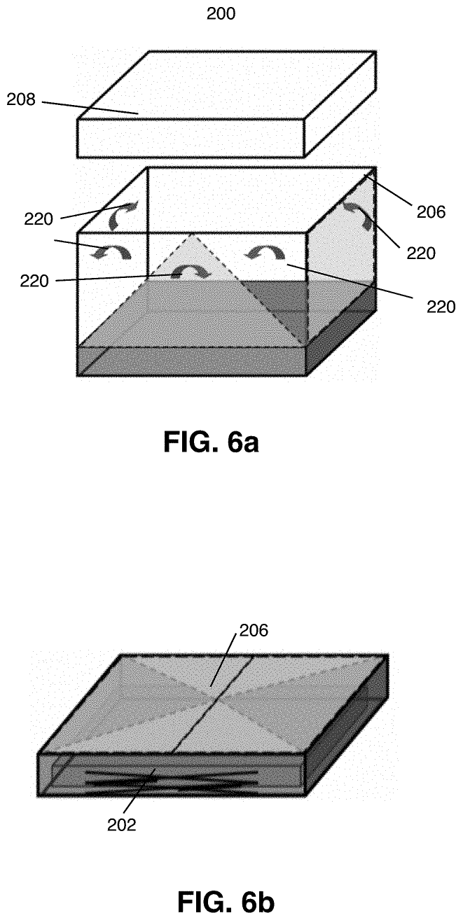

In yet another embodiment, FIGS. 6a and 6b show a box-shaped device 200 that in collapses in a different way. In particular, rigid panel walls of square container 206 “fold” inwardly following an order and direction of arrows 220 placed on the outer walls of container 206 shown in FIG. 6a. This when collapsed, as seen in FIG. 6b, container 206 and UFC 202 are compressed down into a small volume, ready for lid 208 to be placed thereon for closing, storage and transport.

The system of the present invention is simple to operate. In embodiments in which devices are equipped with a timer/digital readout (as in timer/readout 48 of FIGS. 1a-1c and timer/readout 120 of FIGS. 5a-5c, a user may set a time of a disinfection cycle, and may view how much time is left in a cycle. Many variations and options within the scope of the present invention are possible in order to enhance the usability of the device 1. For example, the buckling mechanism can have an auto-lock safety feature such that the device cannot be opened while running, and that unlocks when done and safe to remove the object disinfected. Optionally, a small window may also be included so a user can “see” that it's operating. Although UV light is invisible blue/purple colored LEDs may be added for visual effect.

While embodiments of the invention have been illustrated and described, it is not intended that these embodiments illustrate and describe all possible forms of the invention. Various changes, modifications, and alterations in the teachings of the present invention may be contemplated by those skilled in the art without departing from the intended spirit and scope thereof. It is intended that the present invention encompass such changes and modifications.

What is claimed is:

1. A collapsible, UV disinfecting framework, comprising:
a. an expandable and collapsible framework structure defining, when in an expanded state, an inner space into which an object for disinfection may be placed; and
b. a UV-emitting light source attached to the framework structure, the source positioned such that, when electrically powered, directs ultraviolet energy toward the inner space.

2. The framework of claim 1, wherein the framework structure is a helically shaped compression spring.

3. The framework of claim 1, wherein the framework structure comprises at least one collapsible hinged lattice structure for assembly on an inner wall of a collapsible container.

4. The framework of claim 3, wherein the framework structure comprises four (4) lattice structures, each for assembly on an inner wall of a four walled, substantially square, collapsible container.

5. The framework of claim 1, wherein the UV-emitting light source comprises at least two LED UVC radiating emitters.

6. The framework of claim 5, wherein the UV-emitting light source comprises a string of electrically connected UVC emitters, the string disposed along at least a portion of the framework structure.

7. The framework of claim 1, wherein the UV-emitting light source comprises at least one pod station connected to the framework structure and a UV pod removably connected to the pod station, such that when powered, the pod station supplies electrical power to the connected pod.

8. The framework of claim 1, wherein the UV-emitting light source includes at least two pod stations connected to the framework structure and a respective UV pod removably connected to each pod station, such that when powered, each pod station supplies electrical power to its connected UV pod.

9. A sealable and portable UV disinfecting device, comprising:
a. a framework having a vertically expandable and collapsible framework support that when in an expanded state defines an inner space into which an object for disinfection may be placed, and an electrically powered UV emitter attached to the support that when powered directs energy toward the inner space; and
b. a sealable container that contains the framework and that is collapsible with the framework support.

10. A portable UV disinfecting device, comprising:
a. a UV disinfecting collapsible framework having an expandable and collapsible framework structure defining, when in an expanded state, an inner space into which the object may be placed, and a UV-emitting light source attached to the framework structure, with the source positioned such that, when electrically powered, directs ultraviolet energy toward the inner space;
b. a collapsible container that houses the framework and that is supported by the framework, the container having a closed bottom end and an open top end; and
c. a lid for sealably closing the open end of the container after the object to be disinfected is placed within the container.

11. The device of claim 10, wherein the lid includes a UVC radiating emitter attached to its inner side, and positioned such that, when electrically powered, directs ultraviolet energy toward the inner space.

12. The device of claim 10 further including a platform placed at the bottom of the container onto which the object to be disinfected may be placed,

13. The device of claim 12, wherein the platform includes a UVC radiating emitter positioned such that, when powered, directs is ultraviolet energy toward the inner space.

14. The device of claim 10, wherein the collapsible framework is helically shaped and the collapsible container is cylindrically shaped.

15. A method of disinfecting an object with UV radiation, comprising:
a. expanding a collapsed, open-ended, UVC disinfecting device comprising,
i. a framework having an expandable and collapsible framework structure defining, when in an expanded state, an inner space, and a UV-emitting light source attached to the framework structure, with the source positioned such that, when electrically powered, directs ultraviolet energy toward the inner space; and
ii. a collapsible container that houses the framework and that has an open end;
b. placing the object inside the inner space of the device;
c. sealing the device with a lid place over the open end of the container; and
d. electrically powering the UV-emitting light source.

16. The method of claim 15, wherein the step of electrically powering the UV-emitting light source includes powering the source for a minimum amount of time.

17. The method of claim 16, wherein the minimum amount of time is at least 30 seconds.

18. The method of claim 15, wherein the step of electrically powering the UV-emitting light source includes powering the source for a predetermined amount of time.
